(19) United States
(12) Patent Application Publication
(10) Pub. No.: US 2007/0109450 A1
(43) Pub. Date: May 17, 2007

(54) Signal processing apparatus and control method thereof

(75) Inventor: Seung-jun Lee, Yongin-si (KR)

Correspondence Address: SUGHRUE MION, PLLC, 2100 PENNSYLVANIA AVENUE, N.W., SUITE 800, WASHINGTON, DC, 20037, US

(73) Assignee: SAMSUNG ELECTRONICS CO., LTD.

(21) Appl. No.: 11/600,122

(22) Filed: Nov. 16, 2006

(30) Foreign Application Priority Data
Nov. 16, 2005 (KR) 10-2005-0109643

(51) Int. Cl. H04N 5/44; H04N 5/63

(52) U.S. Cl. 348/730; 348/725

(57) ABSTRACT

A signal processing apparatus and a control method thereof are provided. The signal processing apparatus includes a signal receiver which receives a video signal; a video processor which processes the video signal received by the signal receiver; a display which displays the video signal processed by the video processor; a main controller which determines whether the signal processing apparatus is powered on and initializes the signal receiver and the video processor; and a sub controller which initializes the display while the main controller initializes at least one of the signal receiver and the video processor.

### CROSS-REFERENCE TO RELATED APPLICATIONS

This application claims priority from Korean Patent Application No. 2005-0109643, filed on Nov. 16, 2005 in the Korean Intellectual Property Office, the disclosure of which is incorporated herein by reference.

### BACKGROUND OF THE INVENTION

1. Field of the Invention

The present invention relates to a signal processing apparatus and a control method thereof, and more particularly, to a signal processing apparatus for improving the convenience of a user and a control method thereof.

2. Description of the Related Art

A signal processing apparatus, such as a liquid crystal display (LCD) television (TV), receives a video signal and/or an audio signal from a broadcasting station or an external device such as a video cassette recorder (VCR), a digital versatile disk (DVD) player, a personal computer (PC) and so on, processes the received video signal and/or the received audio signal, and then outputs the processed video signal and/or audio signal.

FIG. 1 is a schematic block diagram of a related art signal processing apparatus 1. The signal processing apparatus 1 includes a signal receiver 11, a decoder 12, a scaler 13, a display 14, an analog-to-digital (AD) converter 15, a digital video receiver 16, an audio processor 17, a signal amplifier 18, an audio output unit 19, and a controller 20. The signal receiver 11 receives a radio frequency (RF) signal. The decoder 12 receives an analog video signal from the signal receiver 11 and outputs a digital video signal. The AD converter 15 converts the received analog video signal, such as “Component” or “HVRGB”, to a digital video signal. The digital video receiver 16 receives a digital video signal such as “DVI/HDMI” and demultiplexes the received digital video signal. The scaler 13 receives the video signal from at least one of the decoder 12, the AD converter 15 and the digital video receiver 16, adjusts resolution of the video according to a characteristic of the display 14 and a user's setting, and outputs the video signal having the adjusted resolution to the display 14. The audio processor 17 receives the audio signal from the signal receiver 11 and decodes the received audio signal, or outputs a received “Audio L/R” signal. The signal amplifier 18 amplifies the audio signal output from the audio processor 17. The audio output unit 19 outputs the audio based on the audio signal received from the signal amplifier 18. The controller 20 controls the overall operation of the components.

As constructed above, the components of the signal processing apparatus 1 can be implemented using integrated circuits (ICs). When the signal processing apparatus 1 is powered on, the components need to be initialized. For example, the components need to be reset by hardware and/or software. Particularly, the signal receiver 11 requires initialization of “phase locked loop (PLL)”, “charge pump” and so on. The decoder 12 requires initialization of “input port switching”, “clamping”, “analog-to-digital converter (ADC) setup” and so on. In many cases, it takes much time for the components to initialize. Especially, when the display 14 is implemented using a LCD panel, after power is supplied to the panel, initialization of backlight (not shown) turn-on, pulse width modulation (PWM) dimming, analog dimming and so on takes a great deal of time in comparison to other components.

However, in the related art signal processing apparatus 1, since the sole controller 20 takes charge of controlling of the other components, there is a disadvantage in that only one command is executed at a time. Thus, upon confirming the powered-on of the signal processing apparatus 1, the controller 20 initializes the components one by one according to a predetermined order. As a result, it takes a considerable time to display the video on a screen after completing the initialization of all the components.

### SUMMARY OF THE INVENTION

The present invention provides a signal processing apparatus for improving the convenience of a user by shortening an initialization time, and a control method of the signal processing apparatus.

According to an aspect of the present invention, there is provided a signal processing apparatus including a signal receiver which receives a video signal; a video processor which processes the video signal received by the signal receiver; a display which displays a video based on the video signal processed by the video processor; a main controller which determines whether the signal processing apparatus is powered on and initializes the signal receiver and the video processor; and a sub controller which initializes the display while the main controller initializes at least one of the signal receiver and the video processor.

The main controller first initializes one of the signal receiver and the video processor having a longest initialization time. The video processor includes a decoder which decodes the video signal received by the signal receiver. The video processor includes a scaler which scales the video signal received by the signal receiver.

The signal receiver further receives an audio signal, and the apparatus further includes an audio processor which processes the audio signal received by the signal receiver. The signal processing apparatus further includes a signal amplifier which amplifies the audio signal processed by the audio processor. The signal processing apparatus further includes an audio output unit which outputs the audio signal amplified by the signal amplifier. The sub controller initializes at least one of the audio processor and the signal amplifier while the main controller initializes at least one of the signal receiver and the video processor.

The sub controller initializes the display, the audio processor, and the signal amplifier in an order based on initialization times of the display, the audio processor, and the signal amplifier. The sub controller controls the audio processor to process the audio signal which is received by and output from the signal receiver.

According to another aspect of the present invention, there is provided a control method of a signal processing apparatus comprising a signal receiver which receives a video signal, a video processor which processes the video signal received by the signal receiver, and a display which displays the video signal processed by the video processor, the method including determining whether the signal processing apparatus is powered on; initializing the signal receiver and the video processor if it is determined that the signal processing apparatus is powered on; and initializing the display while initializing at least one of the signal receiver and the video processor.

The initializing the signal receiver and the video processor includes first initializing one of the signal receiver and the video processor having a longest initialization time.

The signal receiver further receives an audio signal, the signal processing apparatus further includes an audio processor which processes the audio signal received by the signal receiver, and a signal amplifier which amplifies the audio signal processed by the audio processor, and the control method of the signal processing apparatus further includes initializing at least one of the audio processor and the signal amplifier while initializing at least one of the signal receiver and the video processor.

The initializing the display, the audio processor, and the signal amplifier includes first initializing one of the display, the audio processor, and the signal amplifier having a longest initialization time.

The control method further includes controlling the audio processor to process the audio signal received by and output from the signal receiver.

### BRIEF DESCRIPTION OF THE DRAWINGS

The above and/or other aspects of the prevent invention will become apparent and more readily appreciated from the following description of the exemplary embodiments, taken in conjunction with the accompany drawings, in which:

FIG. 1 is a block diagram of a related art signal processing apparatus;

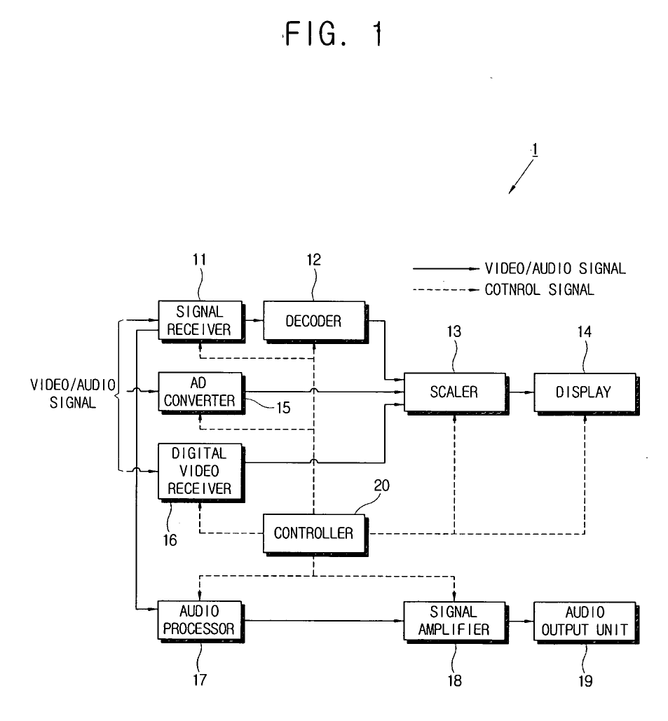

FIG. 2 is a schematic block diagram of a signal processing apparatus according to an exemplary embodiment of the present invention; and

FIG. 3 is a flow chart illustrating a control method of the signal processing apparatus according to an exemplary embodiment of the present invention.

### DETAILED DESCRIPTION OF THE ILLUSTRATIVE, NON-LIMITING EMBODIMENTS OF THE INVENTION

Reference will now be made in detail to the exemplary embodiments of the present invention, examples of which are illustrated in the accompanying drawings, wherein like reference numerals refer to like elements throughout. The exemplary embodiments are described below so as to explain the present invention by referring to the figures.

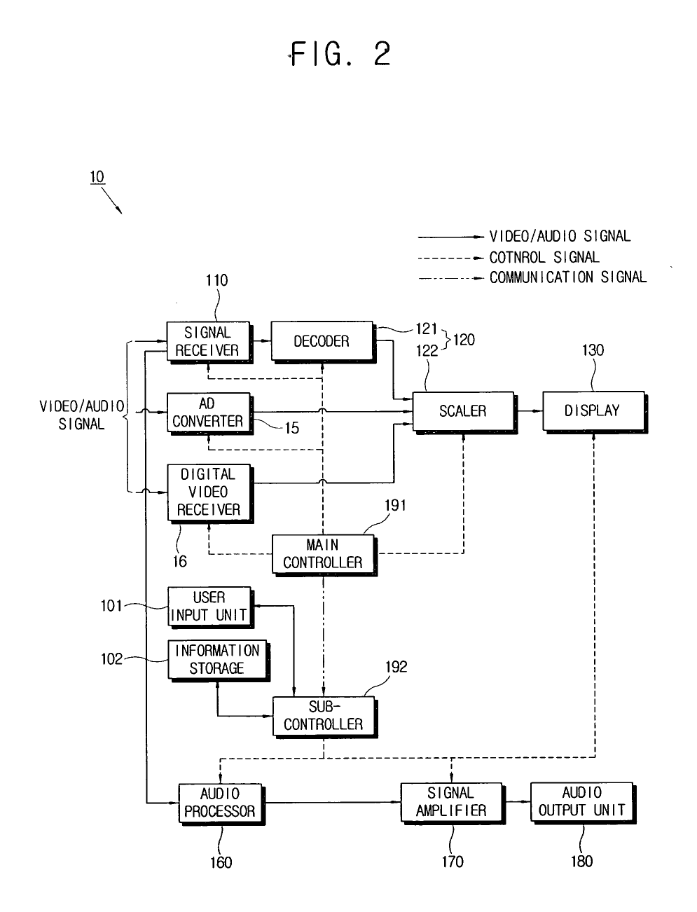

FIG. 2 is a schematic block diagram of a signal processing apparatus 10 according to an exemplary embodiment of the present invention. The signal processing apparatus 10 may be a digital TV such as an LCD TV, a plasma display panel (PDP) TV, a digital light processing (DLP) TV and the like. The signal processing apparatus 10 receives a video signal and/or an audio signal from a broadcasting station or an external device such as a VCR, a DVD apparatus, a PC and so on, processes the received video signal and/or the received audio signal, and then outputs the processed video signal and/or audio signal. The signal processing apparatus 10 has two controllers to initialize two or more components in parallel. Therefore, the convenience of a user can be enhanced by shortening an initialization time.

As shown in FIG. 2, the signal processing apparatus 10 includes a signal receiver 110, a video processor 120, a display 130, a main controller 191, and a sub controller 192. The signal receiver 110 receives a radio frequency (RF) video signal. The signal receiver 110 tunes the video signal to a frequency corresponding to a channel selected by a user under control of the main controller 191. The video signal output from the signal receiver 110 may carry a video in an intermediate frequency such as a picture intermediate frequency (PIF).

The video processor 120 receives the video signal from the signal receiver 110 and processes the video signal. The video processor 120 includes a decoder 121 and a scaler 122. The decoder 121 receives an analog video signal from the signal receiver 110 and outputs a digital video signal by performing analog-to-digital (AD) conversion and decoding for the analog video signal.

The scaler 122 receives the video signal from the decoder 121, adjusts resolution of the video according to a characteristic of the display 130 and a user's setting, and outputs the video signal having the adjusted resolution to the display 130.

The display 130 receives the video signal from the scaler 122 and displays the video based on the received video signal. The display 130 may include an LCD panel, a PDP panel, or the like according to the form of display.

The main controller 191 controls the signal receiver 110 and the video processor 120. The main controller 191 initializes the signal receiver 110 and the video processor 120 by checking whether the signal processing apparatus 10 is powered on by the user. The signal processing apparatus 10 may further include a user input unit 101 through which the main controller 191 may receive input information from the user. The user input unit 101 may be implemented by a remote controller.

The main controller 191 initializes “PLL”, “charge pump”, “SAW filter”, “AGC” of the signal receiver 110, initializes “input port switching”, “clamping”, “ADC setup”, “output format setup” of the decoder 121, and initializes “input port switching”, “contrast/brightness/color/tint setup” of the scaler 122.

It may be preferable but not necessary that the main controller 191 first initializes a component having a longer initialization time. For instance, provided that the scaler 122 has the longest initialization time among the signal receiver 110, the decoder 122, and the scaler 122, the main controller 191 may first initialize the scaler 122.

Additionally, the controller 191 can initialize the components based on user setup information. By way of example, the main controller 191 can initialize the components corresponding to information relating to the channel number, the volume, and the screen adjustment state at the time when the user is watching just before the signal processing apparatus is powered down. In this situation, the signal processing apparatus 10 may further include an information storage 102 in which the user setup information is preferably stored.

The sub controller 192 initializes the display 130 in parallel with the main controller 191 initializing the other components, while communicating with the main controller 191. In case that the signal processing apparatus 10 is an LCD TV, the sub controller 192 initializes “panel power supply”, “backlight (not shown) turn-on”, “PWM dimming”, “analog dimming” of the display 130.

The signal processing apparatus 10 may further include an audio processor 160, a signal amplifier 170, and an audio output unit 180. The audio processor 160 receives the audio signal containing the audio from the signal receiver 110 and performs the audio decoding with respect to the audio signal, or receives and outputs the “audio L/R” signal. The signal amplifier 170 amplifies the audio signal output from the audio processor 160. The audio output unit 180, such as a speaker or the like, outputs the audio based on the audio signal received from the signal amplifier 170.

The sub controller 192 can initialize the audio processor 160 and the signal amplifier 170, as well as the display 130. Particularly, the sub controller 192 initializes “sound port switching”, “prescale”, “deemphasis”, “equalizer”, “balance”, etc. of the audio processor 160, and “volume”, “mute on/off”, etc. of the signal amplifier 170.

It is preferable but not necessary that the sub controller 192 initializes the components in order of a longest initialization time to a shortest initialization time, similar to the main controller 191.

The signal processing apparatus 10 further includes an AD converter 140 and a digital video receiver 150 and receives and processes a video signal in various formats. The AD converter 140 converts the received analog video signal, such as “component” and “HVRGB”, to the digital video signal. The digital video receiver 150 demultiplexes the received digital video signal such as “DVI/HDMI.” The scaler 122 scales the video signals output from the AD converter 140 and the digital video receiver 150.

After the completion of the initialization, the main controller 191 and the sub controller 192 control the components to receive the video signal and/or the audio signal, and output the video and/or audio with respect to the received signal(s). In more detail, the main controller 191 and the sub controller 192 set up the respective corresponding components. In this case, the main controller 191 and the sub controller 192 perform the component setup based on their respective input signals.

For example, the main controller 191 sets up “PLL”, “charge pump”, “SAW filter”, and “AGC” of the signal receiver 110, “input signal standard recognition”, “filter setup”, and “mixer setup” of the decoder 121, and “capture/display setup” and “scaling coefficient setup” of the scaler 122, and the sub controller 192 sets up “input signal standard recognition” and “filter setup” of the audio processor 160.

At this time, the sub controller 192 checks whether the setup of the signal receiver 110 is completed using the information received from the main controller 191, and then sets up the audio processor 160 based on the audio signal output from the signal receiver 110. The audio signal output from the signal receiver 110 may contain the audio in an intermediate frequency such as a sound intermediate frequency (SIF).

It is noted that the main controller 191 and the sub controller 192 can be implemented by computer programs executed by a microprocessor such as a central processing unit (CPU).

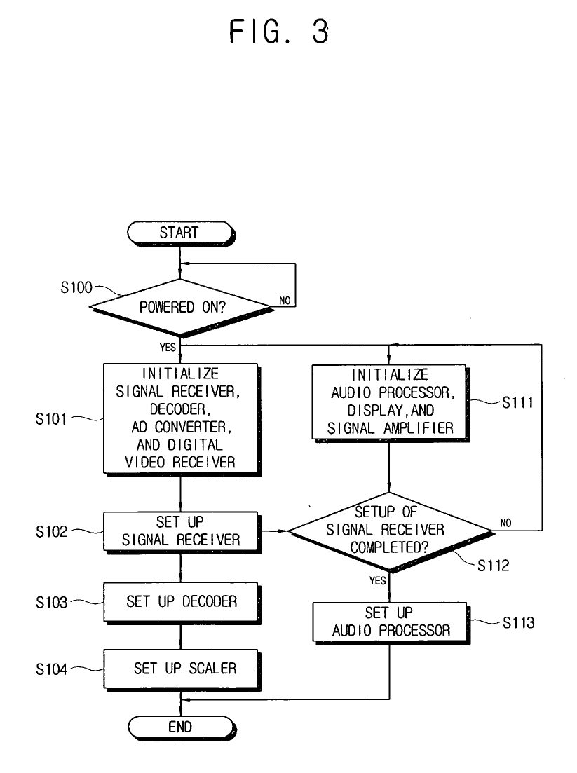

FIG. 3 is a flow chart illustrating a control method of the signal processing apparatus 10 according to an exemplary embodiment of the present invention. First, it is determined whether the signal processing apparatus 10 is powered on by the user at operation S100. If it is determined that the signal processing apparatus 10 is powered on, as a unit of the main control, the signal receiver 110, the decoder 121, the AD converter 140, and the digital video receiver 150 of the signal processing apparatus 150 are initialized at operation S101. As a unit of the sub-control in temporal-parallel with the main control, the audio processor 160, the display 130, and the signal amplifier 170 of the signal processing apparatus 10 are initialized at operation S111.

Upon completing the initialization of the signal receiver 110, the decoder 121, the AD converter 140, and the digital video receiver 150, as a unit of the main control, the signal receiver 110 is set up based on the input video signal at operation S102. Upon completing the setup of the signal receiver 110, the decoder 121 is set up based on the video signal output from the signal receiver 110 at operation S103. After the setup of the decoder 121, the scaler 122 is set up based on the video signal output from the decoder 141 at operation S104.

Finally, as a unit of the sub-control, it is determined whether the setup of the signal receiver 110 is completed at operation S112. If it is determined that the set up of the signal receiver 110 is completed, the audio processor 160 is set up based on the audio signal output from the signal receiver 110 at operation S113.

Accordingly, the present invention provides a signal processing apparatus for improving the convenience of a user by shortening an initialization time, and a control method thereof.

Although a few exemplary embodiments of the present invention have been shown and described, it will be appreciated by those skilled in the art that changes may be made in these embodiments without departing from the principles and spirit of the invention, the scope of which is defined in the appended claims and their equivalents.

What is claimed is:

1. A signal processing apparatus comprising:
a signal receiver which receives a video signal;
a video processor which processes the video signal received by the signal receiver;
a display which displays a video based on the video signal processed in the video processor;
a main controller which determines whether the signal processing apparatus is powered on and initializes the signal receiver and the video processor; and
a sub controller which initializes the display while the main controller initializes at least one of the signal receiver and the video processor.

2. The signal processing apparatus according to claim 1, wherein the main controller first initializes one of the signal receiver and the video processor having a longest initialization time.

3. The signal processing apparatus according to claim 1, wherein the video processor comprises a decoder which decodes the video signal received by the signal receiver.

4. The signal processing apparatus according to claim 1, wherein the video processor comprises a scaler which scales the video signal received by the signal receiver.

5. The signal processing apparatus according to claim 1, wherein the signal receiver further receives an audio signal, and
wherein the apparatus further comprises an audio processor which processes the audio signal received by the signal receiver.

6. The signal processing apparatus according to claim 5, further comprising a signal amplifier which amplifies the audio signal processed by the audio processor.

7. The signal processing apparatus according to claim 6, further comprising an audio output unit which outputs the audio signal amplified by the signal amplifier.

8. The signal processing apparatus according to claim 6, wherein the sub controller initializes at least one of the audio processor and the signal amplifier while the main controller initializes at least one of the signal receiver and the video processor.

9. The signal processing apparatus according to claim 8, wherein the sub controller initializes the display, the audio processor, and the signal amplifier in an order based on initialization times of the display, the audio processor, and the signal amplifier.

10. The signal processing apparatus according to claim 5, wherein the sub controller controls the audio processor to process the audio signal which is received by and output from the signal receiver.

11. The signal processing apparatus according to claim 2, wherein the video processor comprises a decoder which decodes the video signal received by the signal receiver.

12. The signal processing apparatus according to claim 2, wherein the video processor comprises a scaler which scales the video signal received by the signal receiver.

13. The signal processing apparatus according to claim 2, wherein the signal receiver further receives an audio signal, and
wherein the apparatus further comprises an audio processor which processes the audio signal received by the signal receiver.

14. The signal processing apparatus according to claim 13, further comprising a signal amplifier which amplifies the audio signal processed by the audio processor.

15. The signal processing apparatus according to claim 14, further comprising an audio output unit which outputs the audio signal amplified by the signal amplifier.

16. The signal processing apparatus according to claim 14, wherein the sub controller initializes at least one of the audio processor and the signal amplifier while the main controller initializes at least one of the signal receiver and the video processor.

17. The signal processing apparatus according to claim 16, wherein the sub controller initializes the display, the audio processor, and the signal amplifier in an order based on initialization times of the display, the audio processor, and the signal amplifier.

18. The signal processing apparatus according to claim 13, wherein the sub controller controls the audio processor to process the audio signal which is received by and output from the signal receiver.

19. A control method of a signal processing apparatus comprising a signal receiver which receives a video signal, a video processor which processes the video signal received by the signal receiver, and a display which displays the video signal processed by the video processor, the method comprising:
determining whether the signal processing apparatus is powered on;
initializing the signal receiver and the video processor if it is determined that the signal processing apparatus is powered on; and
initializing the display while initializing at least one of the signal receiver and the video processor.

20. The control method according to claim 19, wherein the initializing the signal receiver and the video processor comprises first initializing one of the signal receiver and the video processor having a longest initialization time.

21. The control method according to claim 19, wherein the signal receiver further receives an audio signal,
wherein the signal processing apparatus further comprises an audio processor which processes the audio signal received by the signal receiver, and a signal amplifier which amplifies the audio signal processed by the audio processor, and
wherein the control method further comprises initializing at least one of the audio processor and the signal amplifier while initializing the signal receiver and the video processor.

22. The control method according to claim 21, wherein the initializing the display, the audio processor, and the signal amplifier comprises first initializing one of the display, the audio processor, and the signal amplifier, having a longest initialization time.

23. The control method according to claim 21, further comprising controlling the audio processor to process the audio signal received in and output from the signal receiver.

24. The control method according to claim 20, wherein the signal receiver further receives an audio signal,
wherein the signal processing apparatus further comprises an audio processor which processes the audio signal received by the signal receiver, and a signal amplifier which amplifies the audio signal processed by the audio processor, and
wherein the control method further comprises initializing at least one of the audio processor and the signal amplifier while initializing at least one of the signal receiver and the video processor.

25. The control method according to claim 24, wherein the initializing the display, the audio processor, and the signal amplifier comprises first initializing one of the display, the audio processor, and the signal amplifier having a longest initialization time.

26. The control method according to claim 24, further comprising controlling the audio processor to process the audio signal received by and output from the signal receiver.
